(19) United States
(12) Patent Application Publication
(10) Pub. No.: US 2018/0344154 A1
(43) Pub. Date: Dec. 6, 2018

(54) WIDE ANGLE STEREOSCOPIC FUNDUSCOPY

(71) Applicants: EDUARDO SVETLIZA, CAESAREA (IL); YIGAL DAHAN, CAESAREA (IL); ODED ANNER, CAESAREA (IL)

(72) Inventors: EDUARDO SVETLIZA, CAESAREA (IL); YIGAL DAHAN, CAESAREA (IL); ODED ANNER, CAESAREA (IL)

(21) Appl. No.: 15/757,950

(22) PCT Filed: Feb. 12, 2017
(86) PCT No.: PCT/IL2017/050171

§ 371 Date: Mar. 6, 2018

Related U.S. Application Data
(60) Provisional application No. 62/293,792, filed on Feb. 11, 2016.

(51) Int. Cl. A61B 3/12; A61B 3/14; G02B 27/10; A61B 3/13; A61B 3/00

(52) U.S. Cl. CPC A61B 3/1208; A61B 3/14; A61B 3/0025; A61B 3/132; G02B 27/10

(57) ABSTRACT

An imaging system for wide angle viewing and 3D imaging of the eye fundus in real time comprising: (1) an objective lens system collecting illumination light beam or fluorescent light beam reflected or emitted from the eye fundus, (2) a beam splitting system accepting the light beam from the objective lens system and splitting the light beam into multiple light beams each of which is characterized by a different viewing angle of the fundus, (3) multiple imaging lens systems each of which accepting one of the multiple light beams, (4) multiple band pass filters each of which enabling the view of one of the multiple light beams received from the imaging lens systems, (5) multiple image capturing units each of which captures images transmitted via each one of the multiple light beams and (6) a computer for receiving and combining the images to a single 3D image.

### FIELD OF THE INVENTION

The present invention relates to an imaging system for use as an ophthalmoscope, a retinal camera or a surgery microscope. More particularly, the present invention relates to a wide angle non-contact fundus imaging system having the capability of stereoscopic viewing.

### BACKGROUND OF THE INVENTION

Conventional fundus cameras are known in the art, and most of them use the same concept of illumination where the light beam is incorporated in the optical path. In fact, the pupil acts as the illuminating and viewing aperture. In this well-known design concept the results are satisfactory, but the field of view is limited to 50-60 degrees maximum coverage.

Laser based devices such as a scanning laser ophthalmoscope may enlarge the field of view. However, such devices are relatively non portable, large, and fairly expensive.

Typical conventional fundus cameras are disclosed in the following references:

U.S. Pat. No. 5,608,472 discloses an eye imaging system having a hand held portable image capture unit connected by cable to a housing. The hand held unit includes a light fiber optic for transmitting light to the eye, imaging and focusing optics, and a charge coupled image device.

U.S. Pat. No. 3,944,341 describes a wide-angle indirect ophthalmoscope containing a contact lens that fits over a cornea and surrounded by two rings of optical fibers for illumination of the eye fundus.

U.S. Pat. No. 4,265,519 discloses a wide-angle indirect ophthalmoscope that enables an operator to view the retina from the posterior pole to the equator as a single image. The ophthalmoscope has an observable field that can include the entire retina.

U.S. Pat. No. 8,836,778 discloses a portable hand-held camera for imaging the fundus of an eye, the camera includes a housing comprising an internal cavity terminating at a forward housing end, a forward lens, and a light source configured to direct light from locations distributed around the perimeter of the forward lens forwardly out of the housing end.

In order to enlarge the field of view and to reach the far periphery in the retina, known systems remove the illumination system from the imaging optical module and a ring shaped light source is brought in tight contact with the cornea around the primary lens. Contact with the cornea in this concept is inevitable, otherwise the lighting around the lens does not penetrate through the pupil and reflections from the cornea invade the image path.

In U.S. Pat. No. 5,966,196 of Eduardo Svetliza, the inventor of the present invention, the compromise between illumination and imaging optics is resolved by illuminating the interior of the eye through the sclera. However, the image acquired through the optical module is limited to a single flat two dimensional image.

Thus, it is an aim of the present invention to provide a cost-efficient, precise, high resolution wide angle, non-contact fundus imaging system having the capability of stereoscopic viewing without any requirement to incorporate illumination optics with imaging optics.

Another aim of the present invention is to provide a portable, handheld, compact, lightweight, and easy to operate imaging system to promote ease of examination while providing a wealth of data normally achieved with much larger and more complex systems.

Yet another aim of the present invention is to provide a system that enables taking stereoscopic images of the fundus in real time without the need to dilate the pupils.

Yet another aim of the present invention is to provide an imaging system that obviates the need for a direct contact with the cornea, thereby simplifying the examination procedure and saving the patient from considerable discomfort.

### SUMMARY OF THE INVENTION

The imaging system of the present invention enables physicians to obtain stereoscopic images of the fundus in real time without the need to dilate the pupils. Also, the imaging system according to the present invention does not require physical contact with the cornea, thereby simplifying the examination procedure and saving the patient from considerable discomfort. The imaging system of the present invention is advantageous over prior-art systems due to the following characteristics:
1. The imaging system is a stand-alone system, separate from the illumination system, and thus, it can obtain a picture from every angle without fear of chromatic aberration.
2. The imaging system enables a wide imaging angle of about 80-120 degrees with no corneal contact or pupil dilation since illumination of the interior of the eye is carried out through the sclera.
3. The imaging system enables high resolution imaging of retinal features that measure between 15-20 microns in diameter.
4. The imaging system provides high resolution 3D imaging capabilities in real time. The imaging system enables easy switching to angiography 3D viewing (3D video) by inserting a single filter in the optical path (in collimated light region to allow only fluorescence light to reach the camera).
5. The imaging system facilitates the identification of retinal findings in primary examinations.
6. The imaging system enables zooming in/out in a 3D view.

In accordance with the present invention, there is provided an imaging system for wide angle viewing and 3D imaging of the eye fundus in real time. The imaging system comprising:
an objective lens system, said objective lens system collecting illumination light beam or fluorescent light beam reflected or emitted from the eye fundus,
a beam splitting system, said beam splitting system accepting the light beam from said objective lens system and splitting the light beam into multiple light beams each of which is characterized by a different viewing angle of the fundus,
multiple imaging lens systems, each one of said multiple imaging lens systems accepting one of said multiple light beams,
multiple band pass filters, each one of said multiple band pass filters enabling the view of one of said multiple light beams received from said imaging lens systems,
multiple image capturing units, each one of said multiple image capturing units captures images transmitted via each one of said multiple light beams and
a computer for receiving and combining said images to a single 3D image.

In accordance with the present invention, there is provided another imaging system for wide angle viewing and 3D imaging of the eye fundus in real time. The imaging system comprising:
an objective lens system, said objective lens system collecting illumination light beam or fluorescent light beam reflected or emitted from the eye, a moving optical element, said moving optical element changing the viewing angle of said objective lens system, and thus enabling the transfer of multiple light beams,
a band pass filter, said band pass filter enabling the view of each one of said multiple light beams,
image capturing unit, said image capturing unit capturing images transmitted via each one of said multiple light beams, and
a computer, said computer receiving and combining said images to a single 3D image, wherein said image capturing unit acquiring images consecutively and transferring said images to said computer.

Furthermore, in accordance with the present invention, anaglyph viewing glasses are used to view the 3D image on the screen of said computer.

Furthermore, in accordance with the present invention, a lenticular screen is used with said computer to view the 3D image.

Furthermore, in accordance with the present invention, the objective lens system collects illumination or fluorescent light reflected or emitted from the eye fundus at a wide viewing angle of about 80-120 degrees.

Furthermore, in accordance with the present invention, the objective lens system enables high resolution imaging of retinal features that measure between 15-20 microns in diameter.

Furthermore, in accordance with the present invention, the objective lens system comprising at least one spherical or aspheric lens.

Furthermore, in accordance with the present invention, the at least one spherical or aspheric lens is made of plastic or glass or any combination thereof.

Furthermore, in accordance with the present invention, the beam splitting system is comprised of prisms, mirrors and lenses or a combination thereof.

Furthermore, in accordance with the present invention, the beam splitting is made of glass, plastic or a combination thereof.

Furthermore, in accordance with the present invention, each one of said two imaging lens systems is comprised of at least one lens.

Furthermore, in accordance with the present invention, at least one lens is made of glass, plastic or a combination thereof.

Furthermore, in accordance with the present invention the image capturing units are selected from board cameras, board camera with two sensors and the like.

### BRIEF DESCRIPTION OF THE DRAWINGS

For a better understanding of the invention with regard to the embodiments thereof, reference is made to the accompanying drawings in which like numerals designate corresponding elements or sections throughout and in which:

FIG. 1 illustrates stereoscopic wide angle imaging system in accordance with the present invention; and

FIG. 2 illustrates apparatus for examination of the eye in accordance with the present invention.

### DETAILED DESCRIPTION OF THE DRAWINGS

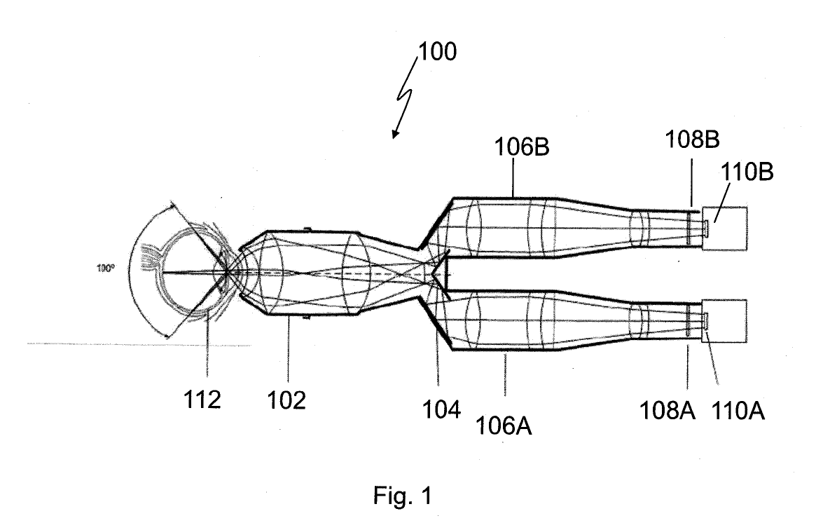

Referring now to FIG. 1, there is shown stereoscopic wide angle imaging system 100 in accordance with the present invention. Imaging system 100 comprising objective lens system 102, beam splitting system 104, two identical imaging lens systems 106A&B, band pass filters 108A&B, and image capturing units 110A&B. Also shown in the figure is cross sectional view of the eye 112.

In accordance with the present invention, objective lens system 102 comprising at least one spherical or aspheric lens made of plastic or glass or any combination thereof.

The objective lens system 102 collects the illumination or fluorescent light reflected or emitted respectively from the retina at a wide viewing angle of about 80-120 degrees and enables high resolution imaging of retinal features that measure between 15-20 microns in diameter.

The objective lens system 102 may contain a single focusing element allowing the physician to obtain a focused image at a wide range of eye types ranging from neonates to adults and under various eye conditions. The focusing element may be automatically or manually controlled.

In accordance with the present invention, beam splitting system 104 may be comprised of prisms, mirrors and lenses or a combination thereof and may be made of glass, plastic or a combination thereof.

Beam splitting system 104 accepts the light from the objective lens system 102 and splits it to two separate beams, each at a different viewing angle of the fundus.

In accordance with the present invention, each one of imaging lens systems 106A and 106B may be comprised of at least one lens that may be spherical or aspheric or a combination thereof and may be made of glass or plastic elements or any combination thereof. Each one of imaging lens systems 106A and 106B accepts a single beam from beam splitting system 104 where each one of these beams transfers an image to each one of image capturing units 110A and 110B.

Imaging lens systems 106A and 106B may contain a relay lens system and aberration correcting lens system as necessary to obtain a high quality distortion free image.

In accordance with the present invention, band pass filters 108A and 108B comprising the form of a filter wheel with one or more mounted filters and are located in front of each one of image capturing units 110A and 110B respectively.

When the fundus is illuminated for viewing, or for excitation for angiography, one of band pass filters 108A and 108B is selected to enable the view of the beam having the required wavelength. Alternatively, the entire wavelength is passed through a non filtering window in filters 108A and 108B to enable the view of the entire spectrum.

Each one of the image capturing units 110A and 110B is placed at the image plane of each one of the imaging lens systems 106A and 106B. Image capturing units 110A and 110B may be fully packaged cameras, board cameras, board camera with two sensors and the like, and it may be CCD or CMOS type cameras (monochrome or color configuration). The dimensions and pixel size of image capturing units 110A and 110B may be selected to comply with the required system magnification and resolution so that the full image is viewed without clipping, and fine features may be viewed without pixelization.

Due to the very wide viewing angle, the image may suffer from increasing distortion and decreasing illumination as one moves away from the image center (zero degree view). However, since the distortion and the illumination can be modeled precisely, it is quite straightforward to correct the image using image processing methods. Since diffuse trans-scleral illumination is used, the retina is illuminated evenly allowing for easy correction of relative illumination issues.

Monochromatic views of the fundus are obtained stereoscopically by illuminating at a specific color with no need for additional filters in the imaging path. 2D full color images may be obtained by illuminating at red, green, and blue (R, G and B) wavelengths, and the two separate images obtained at each color are combined into a single full color image.

Imaging system 100 of the present invention provides a wide angle imaging of the fundus. The light beam reflected or emitted by the fundus is divided into two light beams each transmitting separate images to image capturing units 110A and 110B. Each one of the images consists of a view of the fundus at a slightly different angle as necessary to enable a full stereoscopic view, and the combined images form a single 3D image on the screen (using standard viewing glasses such as active shutter technology).

It should be noted that 3D mono and full color wide images produced at different angles provide more information than any ophthalmoscope/fundus camera in the market.

The obtained images are processed by a computer. Each digital image is rendered using a unique color in an anaglyph color scheme such as red-cyan, red-blue etc. The two digital images are then combined to a single digital image and displayed on the computer screen. The operator/medical practitioner wears a pair of anaglyph viewing glasses having matching color filters to view the respective image on the screen. The two-dimensional view seen without the anaglyph glasses is transformed by the observer's brain to a full color 3 dimensional image upon viewing with the glasses.

The use of a lenticular screen, however, enables 3 dimensional perception without the need for anaglyph glasses or any other aids. Thus, the present invention is not limited to anaglyph viewing and can be used in combination with different kind of technologies offering stereoscopic viewing of the acquired data, notably polarization switching.

Imaging system 100 may be used for stereoscopic angiography using the appropriate excitation wavelength and employing the complementary filters in front of the image capturing units 110A and 110B to selectively view the fluorescence. Each one of the resultant images is given in a different color, and both images are viewed either with anaglyph glasses or via a lenticular screen as described above.

In accordance with the present invention, imaging system 100 is positioned in an enclosure that enables correct optical element positioning, prevents any scattered light from reaching the user, and provides convenient means to hold and position the imaging system 100 with respect to the patient's eye.

In accordance with another embodiment of the present invention, a single imaging path is employed in imaging system 100. This embodiment provides a more compact, lightweight system since imaging lens system 102 does not have to be split into two subsystems. In this case, two images need to be acquired consecutively to enable a stereoscopic viewing. This approach can be realized in a number of alternative ways such as by tilting imaging system 100 with respect to the patient's eye or by incorporating a moving optical element in imaging system 100 which changes the viewing angle of the whole system when moved from side to side. Each one of the above solutions requires a mechanism enabling precise and repeatable movements of imaging system 100 from one angle to another.

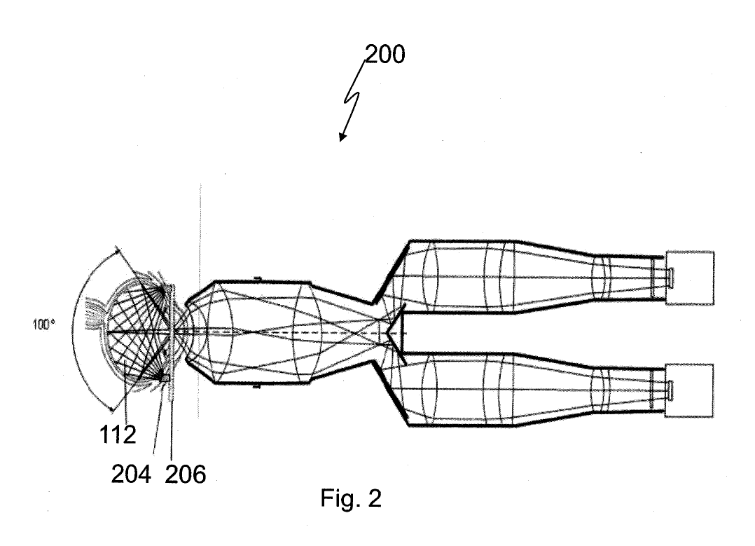

Referring now to FIG. 2, there is shown apparatus 200 for examination of the eye. Apparatus 200 includes the stereoscopic wide angle imaging system 100 of FIG. 1 and a separate illumination system 202. Illumination is performed via the sclera, and imaging is performed through the un-dilated pupil.

Illumination system 202 comprising fiber based ring 206 in contact with or in close proximity to sclera 204. Ring 206 illuminates the retina homogeneously.

1. An imaging system for wide angle viewing and 3D imaging of the eye fundus in real time comprising:
an objective lens system, said objective lens system collecting illumination light beam or fluorescent light beam reflected or emitted from the eye fundus,
a beam splitting system, said beam splitting system accepting the light beam from said objective lens system and splitting the light beam into multiple light beams each of which is characterized by a different viewing angle of the fundus,
multiple imaging lens systems, each one of said multiple imaging lens systems accepting one of said multiple light beams,
multiple band pass filters, each one of said multiple band pass filters enabling the view of one of said multiple light beams received from said imaging lens systems,
multiple image capturing units, each one of said multiple image capturing units captures images transmitted via each one of said multiple light beams and
a computer for receiving and combining said images to a single 3D image.

2. An imaging system for wide angle viewing and 3D imaging of the eye fundus in real time comprising:
an objective lens system, said objective lens system collecting illumination light beam or fluorescent light beam reflected or emitted from the eye,
a moving optical element, said moving optical element changing the viewing angle of said objective lens system, and thus enabling the transfer of multiple light beams,
a band pass filter, said band pass filter enabling the view of each one of said multiple light beams,
image capturing unit, said image capturing unit capturing images transmitted via each one of said multiple light beams, and
a computer, said computer receiving and combining said images to a single 3D image, wherein said image capturing unit acquiring images consecutively and transferring said images to said computer.

3. An imaging system according to each one of claims 1 and 2, wherein anaglyph viewing glasses are used to view the 3D image on the screen of said computer.

4. An imaging system according to each one of claims 1 and 2, wherein a lenticular screen is used with said computer to view the 3D image.

5. An imaging system according to each one of claims 1 and 2, wherein said objective lens system collects illumination or fluorescent light reflected or emitted from the eye fundus at a wide viewing angle of about 80-120 degrees.

6. An imaging system according to each one of claims 1 and 2, wherein said objective lens system enables high resolution imaging of retinal features that measure between 15-20 microns in diameter.

7. An imaging system according to claim 4, wherein said objective lens system comprising at least one spherical or aspheric lens.

8. An imaging system according to claim 5, wherein said at least one spherical or aspheric lens is made of plastic or glass or any combination thereof.

9. An imaging system according to claim 1, wherein said beam splitting system is comprised of prisms, mirrors and lenses or a combination thereof.

10. An imaging system according to claim 1, wherein said beam splitting is made of glass, plastic or a combination thereof.

11. An imaging system according to claim 1, wherein each one of said two imaging lens systems is comprised of at least one lens.

12. An imaging system according to claim 9, wherein said at least one lens is made of glass, plastic or a combination thereof.

13. An imaging system according to claim 1, wherein said image capturing units are selected from board cameras, board camera with two sensors and the like.
